(19) United States
(12) Patent Application Publication
(10) Pub. No.: US 2025/0264474 A1
(43) Pub. Date: Aug. 21, 2025

(54) NEW DROPLET MICRO-TO-MILLI-FLUIDICS-BASED PROCESS TO SCREEN FOR PHENOTYPES OR BIOLOGICAL PROCESSES

(71) Applicants: INSTITUT NATIONAL DE RECHERCHE POUR L’AGRICULTURE, L’ALIMENTATION ET L’ENVIRONNEMENT, PARIS (FR); Centre national de la recherche scientifique, PARIS (FR); Institut National des Sciences Appliquées de Toulouse, TOULOUSE (FR); INSTITUT NATIONAL DE LA SANTE ET DE LA RECHERCHE MEDICALE, PARIS (FR); UNIVERSITE TOULOUSE III-Paul Sabatier, TOULOUSE CEDEX 9 (FR)

(72) Inventors: Gabrielle POTOCKI-VERONESE, LAUTIGNAC (FR); Sophie LAJUS, AYGUESVIVES (FR); Adilya DAGKESAMANSKAYA, TOULOUSE (FR); Amandine DEROITE, COLOMIERS (FR); Delphine LESTRADE, PORTET SUR GARONNE (FR); Rémy FLORES-FLORES, TOULOUSE (FR)

(21) Appl. No.: 18/867,350

(22) PCT Filed: May 17, 2023
(86) PCT No.: PCT/EP2023/063305

§ 371 Date: Nov. 19, 2024

(30) Foreign Application Priority Data
May 20, 2022 (EP) 22305758.9

(51) Int. Cl. G01N 33/58; G01N 33/573

(52) U.S. Cl. CPC G01N 33/582; G01N 33/573; G01N 2333/195; G01N 2333/37

(57) ABSTRACT

A method of screening of at least one phenotype or biological process, for example, self-replicating ability of microbial cells, in a high throughput droplet micro- to milli-fluidic system, based on the use of a photoconvertible or a photoactivable fluorescent protein and artificial intelligence to allow automated imaging and selection.

The present invention concerns a method of screening of at least one phenotype or biological process, for example, self-replicating ability of microbial cells, in a high throughput droplet micro-to-milli-fluidic system, based on the use of a photoconvertible or a photoactivable fluorescent protein and artificial intelligence to allow automated imaging for droplet detection and automated droplet classification.

Microbes represent a gold mine for prospecting and engineering new functions of biotechnolological interest. However, the majority of the species that make up microbial ecosystems are not cultivated yet, and constitute a dark matter whose functioning is difficult or impossible to decipher. Besides, culturomics, as well as strain and enzyme engineering, necessitate fastidious and costly steps of screening to explore a sufficiently large diversity to identify hits.

Here, the inventors of the present invention have developed a new method based on droplet milli-to-micro-fluidics to speed up and miniaturize by several orders of magnitude the process of screening phenotypes or biological processes.

The invention is indeed based on the use of a photoconvertible or a photoactivable fluorescent protein and artificial intelligence to allow automated imaging and selection, at a high rate of droplets per hour, of droplets, followed by their individual sorting, at a high rate of droplets per hour.

The process developed by the inventors could thus be exploited to discover or optimize novel enzymes, metabolic pathways, species and consortia using functional metagenomics, culturomics, enzyme and strain engineering, or even cell-free systems.

It could also be used to screen for i) any cellular phenotypes that are microscopically observable (including those of plant or animal cells encapsulated in droplets of sufficient size), ii) for the production of antimicrobial compounds, and iii) for the synthesis or breakdown of synthetic and natural polymers, as soon as they could be encapsulated in droplets and microscopically observable as molecular assemblies.

The present invention relates to a method of screening of at least one phenotype or biological process, for example, self-replicating ability of microbial cells, in a high throughput droplet micro-to-milli-fluidic system, said method comprising the following steps of:
(a) generating a droplet batch in a carrier fluid to form a plurality of individual bioreactors, each droplet containing a photoconvertible or a photoactivable fluorescent protein and broth media, solution or buffer, at least one of the droplets containing one or several entity(ies),
(b) incubating said droplets over time,
(c) detecting among said droplets at least one droplet of interest, by imaging them,
(d) labelling said detected droplets by switching the fluorescence of the photoconvertible or photoactivable fluorescent protein, wherein said detecting and labelling steps are conducted by an automated electronic processing system, and
(e) selectively recovering the droplets using the label of the droplet.

In particular, said method further comprises:
(f) recovering the entities; and optionally
(g) submitting said entities to biological analysis.

Even more particularly, the method according to the invention comprises repeating steps (a) to (e). More particularly steps (a) to (e) are repeated between 1 and 100 times, for example from 1 to 75, 1 to 60, 1 to 50, 1 to 40, 1 to 30, 1 to 20, 1 to 15, 1 to 10 or 1 to 5 times or once.

By “high throughput droplet milli-to-micro-fluidic system” is meant, in the context of the present invention, one system/one machine that enables formation of droplets (more than 100), at least one measurement on each droplet and the manipulation of the droplets. The manipulation comprises: preparing the droplets, incubation, detection, labelling and sorting the droplet.

The droplet batch is made up of a plurality of droplets of an internal fluid dispersed in a carrier fluid. Each droplet makes up a closed compartment filled with internal fluid, in which chemical or biological reactions may for example occur that may result in revealing the formation or alteration of phenotype or biological process generated by the encapsulated entity.

The droplets generated thanks to the milli-to-microfluidic device are spherical. They are simple (water/oil) or double (water/oil/water) emulsions. The droplet sorting device is either on-chip sorter, or a cytometric cell-sorter, notably FACS. For example, an on-chip sorter may be preferably used for simple water/oil emulsions and a cytometric cell-sorter may be preferably used for double water/oil/water emulsions.

The carrier fluid comprises oil and in particular fluorinated oil containing surfactant for example RAN fluorosurfactant for droplet production.

The internal fluid of each droplet is made up of a base and optionally of entity (ies). The proportions of the entities and the base and/or the natures of the specific entities may vary from one droplet to the next.

The base comprises a broth media, solution or buffer.

By media is meant rich media such as LB or YPD broth or defined medium.

By buffer is meant solution that minimizes change in pH when acid or base is produced.

By solution is meant any aqueous solution containing enzymes, salts and reactants, for example to perform in vitro transcription and translation to produce enzyme variants.

The media, solution or buffer used in the method according to the invention will vary depending on the nature of the entities studied in the droplet bioreactor. The man skilled in the art is able to adapt the media, solution or buffer to the type of entity. For example, growth media such as LB medium, MH medium, defined medium for bacteria, TAP medium for algae, YPD for yeast or any cell culture medium.

The base of each droplet further comprises a photoconvertible or a photoactivable fluorescent protein. Said protein is able to be activated by a labeling device, as will be described later.

In the context of the present invention, the photoconvertible or the photoactivable fluorescent protein (PCFPs) are, respectively, fluorescent proteins whose fluorescence spectra can be shifted by exposure to specific wavelength and proteins with fluorescence that can be switched from a state of non-fluorescent to a state of fluorescent by exposure to specific wavelength.

Examples of such proteins are photoconvertible Dendra2, photoactivable Green Fluorescent Protein (PA-GFP), photoactivable Red Fluorescent Protein (PATagRFP), photoactivable PA-mCherry, photoconvertible Dendra.

In particular, photoconvertible Dendra2, photoactivable Green Fluorescent Protein (PA-GFP) and photoactivable Red Fluorescent Protein (PATagRFP) are contemplated.

PCFPs are encapsulated in a purified form, under the form of a bacterial lysate, or under the form of a microbial cell producing the photoconvertible or photoactivable fluorescent protein.

Entities as described herein covers entities having biological or chemical properties. These entities are responsible for the apparition or the modification of detected phenotype or biological process inside the droplets. These detected phenotype or biological process have a size allowing them to be observable under an optical microscope, that is to say, have a size of at least 0.5 nm, more particularly of at least 500 nm.

The entities that are encapsulated into the droplets can be of the same nature or be different.

For example, depending on the droplet that is considered, the entities can be different or different entities can be encapsulated into the same droplet.

In an alternative embodiment, only the same entity is encapsulated.

It means that the following exemplary cases are covered:
One or several entities are encapsulated in at least one droplet, the entities all being the same one (for example the same bacteria or the same yeast);
One or several entities are encapsulated in at least one droplet, the entities being encapsulated in the same droplet being different (for example: two different bacterial strains);
Several droplets contain different entities (for example one droplet comprises one or more of the bacteria 1 and the next droplet comprises one or more of the bacteria 2, bacteria 1 and 2 being different);
Several droplets contained different entities and the entities encapsulated into one droplet are different (for example, the first droplet comprises one or more of bacteria 1 and one or more of bacteria 2 and the next droplet comprises one or more of bacteria 3 and one or more of bacteria 4);
. . .
Entities covered by the invention are for example self-replicating entities (for example populations or communities of cells).

More specifically, entities having biological properties such as phages, viruses, bacteria, archea, unicellular eukaryote cells (such as yeast, algae, or slime molds), cell lines derived from multicellular eukaryotes (including plants and animals), microorganisms communities, small multicellular organisms, terrestrial fresh water and marine samples, extraterrestrial samples and clinical samples, or cells thereof can be cited.

Self-replicating entities include self-replicating chemistries (such as autocatalytic RNAs).

In one embodiment, said self-replicating entities are asexual self-replicating entities.

Other entities having such as genes, chromosomes, plasmids, fosmids encoding proteins, more specifically enzymes, can also be cited.

In one embodiment, the Poisson distribution can be used to determine the concentration of entities to use to generate droplets in order set the average number of entity (ies) per droplet:

$$p ( k ) = P ( X = k ) = \frac{{λ}^{k}}{k !} e - λ ,$$

in which:
   λ is the average number of entities in one droplet volume, and
   p(k) is the probability to have k entities in a droplet.

More particularly, in the case where the number of entities in the droplets is distributed following a Poisson distribution to obtain only one entity per droplet, there is a probability to obtain some droplets that only encapsulate media, solution or buffer and PCFPs.

In one embodiment of the method according to the invention, at least one droplet of the batch of step a) encapsulates one entity.

In another embodiment of the method according to the invention, each entity is founded by from 1 to 100 entities, in particular from 1 to 5 entities.

In still another embodiment of the method according to the invention, when self-replicating entities are contemplated, said self-replicating entities grow in droplet for between 1 and 20 generations or for between 1 and 25 generations, or more, like 30 or 35 generations for example.

For example, up to 5 generations can be carried out.

In still another embodiment of the method according to the invention, the entities in droplet are incubated for at least one hour until few days, for example for 2, 3, 4, 5, 6, 7, 8, 9, 10 or 20 hours or for 2 or 3 days.

A droplet can thus encapsulate one or more entities, for example, 1, 2, 3, 4, 5, 6, 7, 8, 9, 10, 15, 20, 25, 30, 35, 40, 45, 50, etc. The maximum number is dictated by the maximum carrying capacity of the media, solution or buffer for the chosen entity.

In particular, part of the generated droplets can be incubated at controlled temperature.

Still particularly, droplets can be kept static or moving in one single tube, called an incubation tube. Droplets are incubated in the incubating tube to allow biochemical and/or biological reactions.

Said droplet batch can contain a various number of droplets.

In particular, it contains between 100 and 10¹⁰ droplets, more particularly between 100 and 10⁸ or between 10⁴ and 10⁶.

Still particularly, the volume of each droplet is from pL to μL, more particularly from 0.5 to 20 pL, even more particularly from 1 to 8 pL, for example of about 3 pL.

Still particularly, the droplets are chosen from water/oil/water (w/o/w) double-emulsion droplets and water/oil (w/o) single-emulsion droplets.

By “screening of at least one phenotype or biological process” is meant in particular, the screening of any cellular or colony phenotypes or biological processes that are observable with an optical microscope (including those of plant or animal cells encapsulated in droplets of sufficient size).

The optimization of novel enzymes, metabolic pathways, species and consortia using functional metagenomics, culturomics, enzyme and strain engineering, or even cell-free systems are particularly contemplated as well as the screening for the production of antimicrobial compounds, or for the synthesis or breakdown of synthetic and natural polymers, as soon as they could be encapsulated in droplets and microscopically observable as molecular assemblies.

As a consequence the following examples can be cited:
bacteria or yeasts (in particular non-fluorescent ones) screened for intracellular or extracellular enzymes, or catabolic pathways resulting in cell growth on any kind of substrates, in particular natural ones, that are non-chromogenic or non-fluorogenic, shape or size of cells after growth (hyphae/not, for example, for some yeasts)
modification of droplet size, due to a biotic or abiotic effect on the droplet content (for example due to bacterial growth),
no growth of the encapsulated strain or of a co-encapsulated strain,
“mixed growth” of two microorganisms (bacteria and yeast for example),
production or accumulation of an extra- or intra-cellular non-soluble product (polymer, lipid),
production or breakdown of a polymeric substrate.

The system for screening comprises a support defining an observation chamber for receiving the droplet batch.

The system further comprises an automated electronic processing system configured to detect specific droplets of the droplet batch, also called droplets of interest, i.e. a droplet encapsulating the entity (ies) of interest which is the entity initially encapsulated in the droplet that is responsible for the desired phenotype or that executed a specific biological process for which the screening is conducting, and to label said detected droplets.

The system further comprises a recovery device.

Advantageously, the system further comprises motorized means for moving the support relative to the processing system.

For example, the support is a rectangular block extended along a longitudinal axis X and a transverse axis Y perpendicular to the longitudinal axis X. Furthermore, the support has a thickness along an elevation axis Z perpendicular to the longitudinal axis X and the transverse axis Y.

Possibly, the support is a multi-well plate. For example, the support comprises between 1 and 96 wells.

The observation chamber is configured to store the droplet batch during a detecting step and a labeling step. The droplets are advantageously distributed in a two-dimensional layer along the plane of the observation chamber comprising the longitudinal axis X and the transverse axis Y, so that no droplet is superimposed on another droplet.

The observation chamber has a screening region. The screening region is able to contain between 1 and 1000 droplets at the same time, in particular 300 droplets. Furthermore, the dimensions of the screening region are suitable for it to be able to be completely swept by the processing system.

The support is transparent and compatible with optical microscope, at least in the screening region, to allow the detection and labeling of the droplets of interest over the droplets present in the screening region by the processing system.

The processing system comprises a detecting device able to detect the droplets and a labelling device able to label said detected droplets.

The detecting device is able to identify, among the droplets of the droplet batch, which droplets are to be considered as droplets of interest, also called “positive droplets” and which ones are not, the latter being called “negative droplets”.

By “droplet of interest” is meant a droplet encapsulating the entity (ies) of interest. An entity of interest is the entity initially encapsulated in the droplet that is responsible for the desired phenotype or that executed a specific biological process for which the screening is conducting. For example, microbial growth in specific culture media, the production of antimicrobial compounds, or the synthesis or breakdown of synthetic and natural polymers can be cited. For example, in the case of screening for the growth of an entity such as a bacteria, droplets of interest vs negative droplets would be droplets with cell growth vs droplets with no cell growth.

The detecting device is able to image and automatically detect the droplets, and to classify the droplets in predetermined class(es).

The detecting device comprises an imaging unit and a classifying unit.

The imaging unit allows an optical detection of the droplets of the droplet batch.

For example, the imaging unit is an optical microscopy system. The imaging unit comprises a sensor, that is for example able to detect the light transmitted image of droplets in the screening region and a light source set up to illuminate the screening region.

For example, the sensor is a camera able to form an image of the screened region. The obtained image resolution for example makes it possible to identify the droplets edges as well as the phenotype or biological process present in the droplets.

The camera is configured to acquire at least one partial image of the droplet batch. This partial image corresponds to the part of the droplet batch imaged by the optical system for a relative position. Thus, the optical system is arranged for projecting different partial images of the droplet batch onto camera during a two-dimensional scanning of the droplet batch by the optical system.

By “two-dimensional scanning of the droplet batch by the optical system” is meant relative displacement of the optical system in relation to the droplet batch in at least the two directions X and Y.

The imaging unit is also configured to segment the at least one partial image into image portions, each image portion corresponding to a single droplet. In other words, the imaging unit is able to detect droplets in the image.

In a preferred embodiment, the imaging unit is able to apply an edge detector filter to the image, for example using a Canny algorithm, and a circular Hough transform to detect the different droplets.

The classifying unit is able to receive signals from the imaging unit and process them in order to classify the imaged droplets.

For example, the classifying unit includes a memory and a microprocessor. The classifying unit is able to decide the classification of at least one droplet of the droplet batch based on the images obtained during the imaging step.

Classification criteria are for example recorded in the memory. The microprocessor is able to compare images to parameters of the classification criteria.

Advantageously, the microprocessor is configured to implement an artificial intelligence algorithm to classify the droplets.

For example, an input variable of the artificial intelligence algorithm is an image portion of the image taken, and at least one output variable of the artificial intelligence algorithm is an indication relative to a class of the droplet.

The class of the droplet is representative of the observed phenotype or biological process resulting from the entities initially encapsulated within.

For example, the classifying unit is configured to classify the droplets between a first class corresponding to the droplets of interest (positive droplets) and a second class corresponding to the negative droplets. The classifying unit is thus able to classify each droplet as a positive droplet or a negative droplet.

Advantageously, the artificial intelligence algorithm is an artificial neural network.

The artificial neural network used by the classifying unit may include a system trained based on images or samples that have been previously classified by a human expert.

The memory of the classifying unit is able to store determination software configured to determine, via the artificial neural network, in the image taken by the image sensor, the class of each droplet represented in the image. The microprocessor is then able to execute the determination software.

The classifying unit is configured to determine, via the artificial neural network and in the image taken by the camera, the class of each droplet represented in the image, an input variable of the artificial neural network being an image portion depending on the image taken by the sensor, and at least one output variable of the neural network being an indication relative to the class of the droplet.

The neural network includes a plurality of artificial neurons organized in successive layers, i.e., an input layer corresponding to the input variable(s), an output layer corresponding to the output variable(s), and optional intermediate layers also called hidden layers and arranged between the input layer and the output layer.

Each layer takes its inputs from the outputs of the previous layer. Each layer is composed of a plurality of neurons, taking their inputs from the neurons of the previous layer. Each synapse between neurons is associated with a synaptic weight, so that the inputs received by a neuron are multiplied by this weight, then summed by the said neuron.

Advantageously, the artificial neural network algorithm has previously been trained using training data. The training data may include images of droplets and their designated classes which have previously been assigned by an expert. For example, the training data may include image portions classified as corresponding to positive droplets and image portions classified as corresponding to negative droplets.

Advantageously, the training has been done prior to the generation of the droplet batch.

Once the neural network has been trained, the neural network may be used to identify or predict the class of the droplets within an image. For example, an unclassified image is provided to the network and the network outputs a classification. The classification may indicate, for each droplet within the image portion, if said droplet is a positive droplet or a negative droplet.

The artificial neural network is preferably a convolutional neural network (CNN).

In a preferred embodiment, the convolutional neural network takes into input 64 pixels by 64 pixels image portion and output a number chosen between 0 and 1, 1 corresponding to a positive droplet and 0 corresponding to a negative droplet.

The convolutional network is configured to alternate between a convolution layer and a down-sampling layer. For example, the network comprises two convolutional/down-sampling pairs, followed by a fully connected layer that classifies output with one label per node.

Advantageously, the classifying unit is able to store the coordinates of the positive droplets into the memory.

The detecting device is able to communicate the obtained coordinates to the labeling device.

The labeling device is able to label a droplet based on the classification decision of the droplet.

For example, the labeling device comprises a laser able to specifically illuminate a droplet with preset parameters so as to activate the photoconvertible or photoactivable fluorescent protein.

Advantageously, the labeling device is a laser-scanning microscope, notably a laser-scanning confocal microscope.

For example, the labeling device comprises a laser configured to illuminate the positive droplets selectively to switch the fluorescence of the photoconvertible or photoactivable fluorescent protein encapsulated within. The laser has preferably an output wavelength in the range of 300 to 520 nm, for example 405 nm.

The labeling device is able to load the stored coordinates of the positive droplets and, for each coordinate, to create a circular region of interest. The laser is then able to scan these regions of interest and photo-switch the positive droplets.

Advantageously, the labeling device is able to correct droplet coordinates that were stored in the detecting device by correlating a camera image and a confocal image of a same zone, to measure a shift between said camera image and said confocal image and to correct the coordinates, based on the measured shift. These corrected coordinates are then used for creating the regions of interest.

Said camera image and confocal image may be taken and compared in a coordinates correcting step. This correction prevents any misalignment that may occur between the detecting and labeling steps because of different light paths being used in these steps.

Each region of interest is centered on a specific coordinate. Preferably, each region of interest has a diameter smaller than the diameter of a droplet, and is previously determined according to the system.

The recovery device is able to allow a droplet to be recovered based on the labeling. For example, the double water/oil/water emulsions recovery device comprises a cells/particles sorter by flow cytometry or another sorting device. Preferably, the recovery device is a cytometric cell-sorter. FACS (Fluorescence Activated Cell Sorting) can in particular be cited. An on-chip sorting device can also be used.

Recovering of the entities as mentioned in step (f) can be conducted by any method know by the man skilled in the art. For example, incubation of the droplet content in a rich media can be conducted to allow cell multiplication.

In particular, the biological analysis of step (g) comprises but are not limited to the DNA sequencing, taxonomical and functional annotation of the genes, phenotypic characterization or enzymatic activity characterization of the recovered entities.

The man skilled in the art is able to determine how to conduct these analyzes based on its routine work.

A method for screening of at least one phenotype or biological process, for example, self-replicating ability of microbial cells, in a high throughput droplet micro-to-milli-fluidic system will now be described. The system previously described is provided for carrying out said method.

Droplet batch is produced using a micro-to-milli-fluidic device. Advantageously, water/oil or water/oil/water droplets could be prepared. Droplet content is made of media, solution or buffer plus a photoconvertible or a photoactivable fluorescent protein, and eventually one or more than one entity (ies). Droplet batch is then incubated in an incubation tube under optimal conditions to allow the expected reaction or biological process to happen. The incubation tube could directly be within the observation chamber.

The droplet batch is then injected into the observation chamber using an injection device. Alternatively, the screening system is provided directly with the droplet batch in the observation chamber.

Advantageously, in the observation chamber, the droplets are arranged according to a compact arrangement, in particular an arrangement in staggered rows along a plane transverse to the axis Z at least in the screening region. Advantageously, the droplets are arranged in the observation chamber in a single layer, so that no droplet is superimposed on another droplet.

During a detecting step, droplets of interest are detected among the other droplets of the droplet batch.

The detecting step is performed in the observation chamber with substantially no circulation of the droplets.

During the detecting step, the imaging unit, more particularly the sensor of the imaging unit, acquires at least one partial image of the droplet batch including at least one, preferably several droplets.

Each acquired image is then segmented into image portions, each image portion corresponding to a single droplet. Preferably, the segmentation sub-step is done by applying an edge detector filter to the image, for example using a Canny algorithm, and a circular Hough transform to detect the different droplets.

Following the detecting step, the classifying unit classifies the droplets in different classes.

For example, during this classifying step, the image portions are analyzed and compared to the classification criteria. The classifying unit determines to which class each droplet belongs.

For example, during the classifying step, all of the droplets showing the same phenotype or objects resulting from a biological process (for example the numbers of cells) are assigned to the same class by the man skilled in the art. Two or more classes can be defined.

The classification is advantageously automated. Advantageously, the classification is performed by an artificial intelligence algorithm, preferably by an artificial neural network, for example a convolutional neural network.

An input variable of the artificial intelligence neural network is an image portion of the image taken, and at least one output variable of the artificial intelligence algorithm is an indication relative to a class of the droplet.

For each image portion, the classifying unit determines the class associated with the droplet.

The method preferably comprises a preliminary step of training the algorithm. During this preliminary training step, an expert annotates a dataset including images of droplets and their designated classes, then the algorithm is trained on this dataset.

Advantageously, the preliminary step of training the algorithm takes place prior to the step of producing the droplet batch.

In a preferred embodiment, droplets are classified between positive droplets and negative droplets.

The spatial location of each positive droplet, notably its coordinates in the plane of the support, is recorded in the memory of the detecting device.

The droplets are then labeled using the labeling device based on their class.

Advantageously, the droplets are kept immobile in the chamber, for the time needed for the detecting and labeling steps.

During the labeling step, the labeling device loads the coordinates of the positive droplets and defines a region of interest for each coordinate with said coordinate as center of the region.

Advantageously, when different light paths are used for the detecting and the labeling steps (for example with a camera and with confocal head respectively), the labeling device performs a droplet coordinates correcting step before defining the regions of interest. For example, an image obtained by the camera of the detecting device and a confocal image of the same zone obtained by the labeling device are compared and the eventual translational, rotational and/or scaling shift is measured. Then, the measured shift is used to correct the coordinates, which are used for defining the regions of interest.

Switching is then performed by applying illumination, done by the laser, on the protein in the droplet in all regions of interest.

Advantageously, the photo-switching of a droplet is performed by applying one or several iterations of the laser, at the center of the region of interest. The number of iterations is variable depending on the observation conditions, for example, the zoom of the camera or laser power.

After the labeling step, the droplets that have been classified as positive, then labeled, can be distinguished from the other droplets, based on their label.

The detecting steps and labeling steps are then repeated on several positions of the observation chamber to scan the droplet batch and detect and photo-switch positive droplets in the droplet batch.

For example, the support is moved relative to the processing system.

For example, in an embodiment in which the support is a multi-well plate, the support is moved relative to the processing system so that each well defines a position corresponding to a partial image.

Advantageously, the automatic detection of positive droplets and photo-switching is performed at a throughput of more than 36,000 droplets per hour, for example of 91,000 droplets per hour.

A recovery step is then carried out by analyzing the droplet batch by on-chip sorting or by cytometric cell-sorting, notably by FACS. Droplet labelling is then detected to identify the positive and the negative droplet populations. Positive droplets are then individually sorted. Advantageously, it is possible to recover the droplets individually.

The method combines image analysis to identify positive droplets and microscope automation to photo-switch single droplets. Furthermore, this process is automatically repeated on several positions to perform a high content photo-switching experiment.

The detection of positive droplets according to the above-mentioned method is more efficient and less time-consuming than if it was done manually by using an epifluorescence microscope. The use of an automated electronic processing system for the detecting and labelling steps also considerably reduces false positives that would be due to switching of non-positive droplets.

Notably, using an artificial neural network, such as a convolutional neural network, is particularly reliable to prevent false positive. Indeed, the classification algorithm may be easily optimized by adapting the training set to false positive situations such as droplets on top of the detected droplet or “double” droplets (smaller droplet inside the detected droplet). The droplets are thus better characterized before recovery thereof.

Such screening system allows the specific recovery of a population of droplets.

The present invention is illustrated in more detail in the following figures and examples.

### FIGURES

FIG. 1: Simple emulsion (SE) droplets containing 19 μM of pure PCFP stored at −20° C. during 15 days. Switch at 40× during 5 s after 7 days at +4° C. Observation: 10×.

FIG. 2: Characterization of the switch amplitude and stability of the purified Dendra2 protein in (A) microplate (switch conditions: 20 s, 2.5×, directly in the microplate) and (B) in droplets (observation 10×) before switch (C) and after switch (switch conditions: 40 s, 2.5×) using purified Dendra2 (19 μM).

FIG. 3: Characterization of Dendra2 switch amplitude and stability of cell lysate in (A) microplate (switch conditions: 20 s, 2.5×, directly in the microplate) and (B) droplet before switch (C) and after switch (switch conditions: 40 s, 2.5×) using unpurified Dendra2 (lysate).

FIG. 4: Characterization of Dendra2 switch amplitude and stability in expressing bacterial cells in (A) microplate (switch conditions: 40 s, 2.5× directly in the microplate) and (B) droplet before switch (C) and after switch (switch conditions: 40 s, 2.5×) using BL21-pET3-Dendra2 E. coli strain (DO=5).

FIG. 5: Simple emulsion obtained with manual device. (A) 50 μm droplets with pure Dendra2 (19 μM) mixed with droplets of water. (B) Droplets with fluorescein (19 μM) mixed with droplets of water. Day 0: day of droplet production; Day 1: after one day of storage (+4° C.). Observation: 10×.

FIG. 6: Sorting results of a mixture of 50% w/o/w droplets containing unswitched Dendra2 (green) and the control strain and 50% w/o/w droplets containing switched Dendra2 (red) and a positive metagenomic clone. Left panel and central panel correspond to the two applied filters to select singulet droplets only. Right panel corresponds to screening of droplets on green vs red fluorescence intensity.

FIG. 7: Sorting results of a mixture of 98% w/o/w droplets containing unswitched Dendra2 (green) and the negative control strain and 2% w/o/w droplets containing switched Dendra2 (red) and the positive control strain. Left panel and central panel correspond to the two applied filters to select singulet droplets only. Right panel corresponds to screening of droplets on green vs red fluorescence intensity.

FIG. 8: Observation of droplets initially containing maximum one cells at different times and grow on minimum media with different oligosaccharides as carbon sources.

FIG. 9: Observation of yeast strains growth in droplets at different times.

FIG. 10: Set up of switch of individual droplet with confocal microscopy. In the red channel, arrow shows the chosen droplet to be switched before and after laser application. * shows droplets previously switched during the setting up.

FIG. 11: Violin plot representation of the fluorescence intensities, in green and in red, with and without switch of Dendra 2 protein. Intensities were measured in the red channel (A and B) and in the green channel (C and D) at the activation planes (A and C) and at the focus plane (B and D). White representations are for non-switched droplets and grey ones for switched droplets. A minimum of 273 droplets was analysed for each condition.

FIG. 12: FACS density blot results obtained with droplets after wide-field and confocal microscope switching.

FIG. 13: Detection of droplets and bacterial growth classification on transmitted light image. The left column shows the original image, the two middle columns show the detected droplets, black circles for the droplets detected as negative and white circles for droplets detected as positive, respectively and the right column shows the merge image. Top images are obtained with the “canny” method and bottom images with CNN method (right column).

### EXAMPLES

### PCFPs to Label Hit Droplets

The first steps to establish the invention were to construct the PCFP expressing cells and to characterize several PCFPs (fluorescence intensity, stability after switch, storage stability, FACS detection limit, leakage in droplets) under different forms (purified proteins, E. coli cell lysates and expressed in E. coli cells).

### Strain Construction

The pET3-Dendra2, pET3-PA-GFP and pET3-PATagRFP plasmids express His-tagged (6×his tag) versions of the fluorescent proteins under the control of T7 promoter. Corresponding ORF fragments were amplified by PCR (6×his tag coding sequence was added to the forward primers). The resulting fragments were inserted in the Nde1 and BamH1 sites of the pET-3a vector (NOVAGEN). BL21 E. coli cells (New England Biolabs) were transformed with one of those plasmids to produce the fluorescent proteins.

### PCFP Description

Three PCFP were studied: Dendra2, photoactivable Green Fluorescent Protein (PA-GFP) and photoactivable Red Fluorescent Protein (PATagRFP). PCFP description is available in Table 1. The aim was to use a PCFP as droplet label to switch or photoactivate their fluorescence only in the droplets that should be sorted. Practically, switch or photactivation was performed in first instance, using wide-field microscopy with a led coupled with a filter at λex 435±40 nm (BrightLine HC).

TABLE 1
PCFP description

| Dendra2 | (Gurskaya et al., | Green: | From | From |
|---|---|---|---|---|
| | Engineering of a | 490 nm/507 nm | 405 nm | green to red |
| | monomeric | Red: | to 488 nm | |
| | green-to-red | 553 nm/573 nm | | |
| | photoactivatable | | | |
| | fluorescent | | | |
| | protein | | | |
| | induced by blue | | | |
| | light. 24(4), | | | |
| | 461-466) | | | |
| PA-GFP | (Patterson & | Green: | 488 nm | From |
| | Lippincott- | 488 nm/510 nm | | low green to |
| | Schwartz, J. | | | high green |
| | (2002). A | | | |
| | photoactivatable | | | |
| | GFP for | | | |
| | selective | | | |
| | photolabeling of | | | |
| | proteins | | | |
| | and cells. | | | |
| | Science, | | | |
| | 297(5588), | | | |
| | 1873-1877.) | | | |
| PATagRFP | (V. Subach et | Red: | From | From no |
| | al., Bright | 555 nm/584 nm | 488 nm | fluorescence |
| | Monomeric | | to 532 nm | to red |
| | Photoactivatable | | | |
| | Red Fluorescent | | | |
| | Protein for Two- | | | |
| | Color Super- | | | |
| | Resolution | | | |
| | sptPALM of Live | | | |
| | Cells. Journal of | | | |
| | the American | | | |
| | Chemical | | | |
| | Society, | | | |
| | 132(18), 6481- | | | |
| | 6491) | | | |

### PCFP Purification

PCFP were purified using cobalt resins according manufacturer instructions (Fisher Scientific). We then determined the PCFP concentrations using NanoDrop (Table 2). The strains have production rates from 0.24 to 0.35 g of PCFP per liter of cytoplasmic cell extract.

TABLE 2
Amounts of PCFP obtained after purification from E. coli
cell extracts at DO600 nm 80.

| | | | Total | Production |
|---|---|---|---|---|
| | Volume (mL) of | | amount | rate (g of |
| | cell extract | Concentrations | (mg, VF = | PCFP/L of |
| PCFP | (OD600 nm = 80) | (mg/mL) | 3.5 mL) | cell extract) |
| Dendra 2 | 22.5 | 1.52 | 5.3 | 0.24 |
| PA-GFP | 21.6 | 2.13 | 7.5 | 0.35 |
| PATagRFP | 9.0 | 0.63 | 2.2 | 0.25 |

### Purified PCFP Storage Stability

The storage stability of the PCFPs was determined by comparing their fluorescence just after purification and after two weeks stored at −20° C. 19 μM of each PCFP were encapsulated after two weeks storage at −20° C. The emulsions were stored at +4° C. during 7 days and switched at 40× during 5 s on wide-filed microscope.

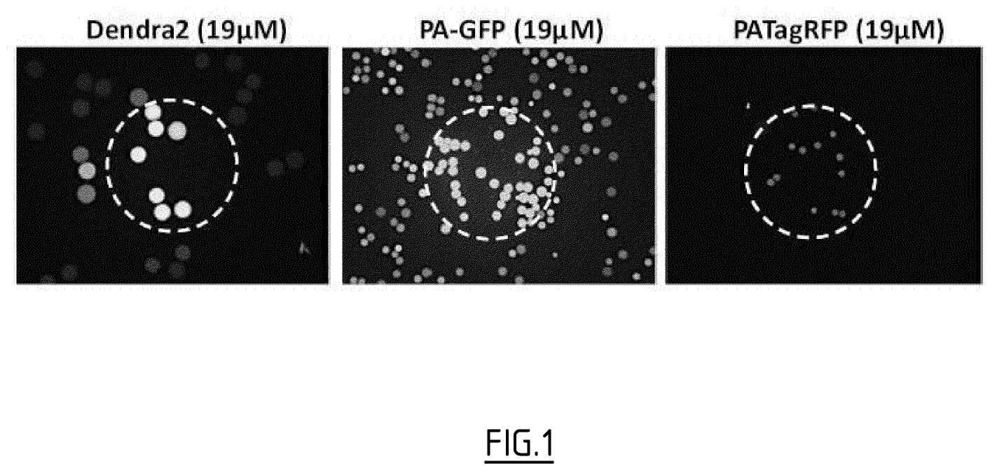

For the three PCFPs, a clear switch was observed even in those drastic conditions (FIG. 1). These results show that it is possible to store the three purified PCFPs without affecting their switchable ability.

PCFP Fluorescence Normalization with that of Fluorescein

In this study, it was wanted to calculate the amount of switched PCFP which was required to sort the droplets. As a reference, as limit of fluorescein detection, 10,000 molecules per 3 pL droplet were used, corresponding to 5.44 nM.

The question was then: what is the switched PCFP concentration leading to the same fluorescence intensity than 5.44 nM of fluorescein?

To answer it, we measured the droplet fluorescence by microscopy. To do that, 19 μM of fluorescein or PCFP were encapsulated in SE droplets. The PCFP were immediately switched at 40× during 30 seconds and the fluorescence intensity was quantified using the ImageJ software (before and after switch). To calculate the level of fluorescence in one droplet, the fluorescence of 10 droplets was measured randomly for each condition, and the mean and the standard deviation were calculated (Table 3).

TABLE 3
Fluorescence level of 19uM fluorescein and PCFP encapsulated into SE
droplets before and after switch (30 s, 40X). The results were obtained
by measuring fluorescence intensity of 10 droplets (randomly
chosen) in order to calculate means and standard deviations.

| | | | | Detection | |
|---|---|---|---|---|---|
| | | | | limit (μM | |
| | | | | of switched | Detection |
| | | | | PCFP, | limit |
| | | | | calculated | (mole- |
| | | | | from the | cules |
| | Fluorescence intensity (a.u.) | | | fluorescence | per |
| Fluorescent | before | after | | after | 18 μm |
| molecule | switch | switch | difference | switch) | droplet) |
| Dendra2 | 24.3 ± 2.7 | 70.9 ± 12.7 | 46.5 | 1.43 X 10⁻³ | 2,623 |
| (red) | | | | | |
| PA-GFP | 10.7 ± 0.5 | 53.0 ± 0.6 | 42.3 | 1.91 X 10⁻³ | 3,509 |
| (green) | | | | | |
| PATagRFP | 22.7 ± 1.9 | 34.1 ± 2.5 | 11.4 | 2.98 X 10⁻³ | 5,471 |
| (red) | | | | | |
| Fluorescein | 18.6 ± 1.7 | / | / | Known | Known |
| (green) | | | | value: | value: |
| | | | | 5.44 X 10⁻³ | 10,000 |

The detection limit for the three PCFPs were thus quite similar (few thousand molecules per droplet).

### Dendra2 Characterization

### Switch Stability

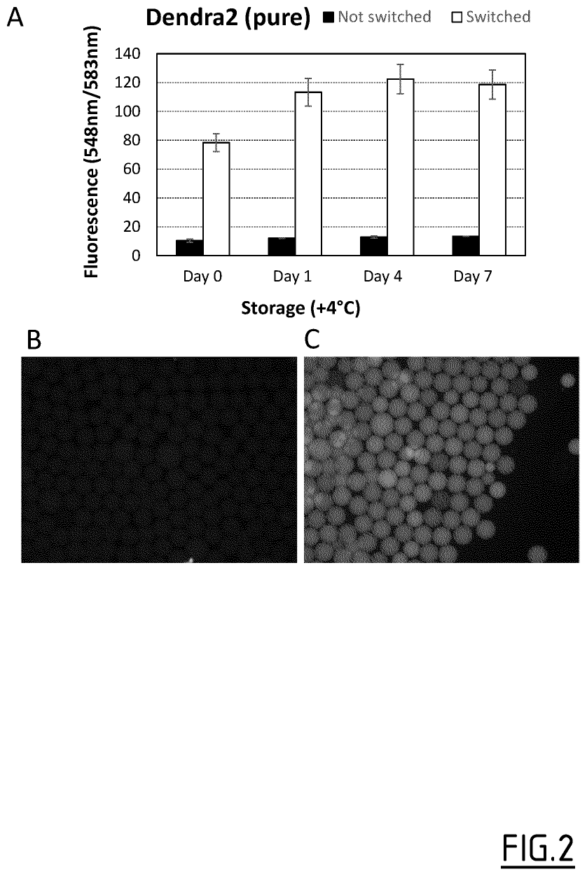

The switch amplitude and stability of Dendra2 was characterized in the microplates (FIG. 2A) and in droplets (FIG. 2B and FIG. 2C). A significant difference of red fluorescence intensity was found before and after switch (7 fold according to the data obtained in microplate (FIG. 2A). Moreover, this fluorescence difference was stable over time (and even accentuated) at least seven days when the emulsion is kept at +4° C.

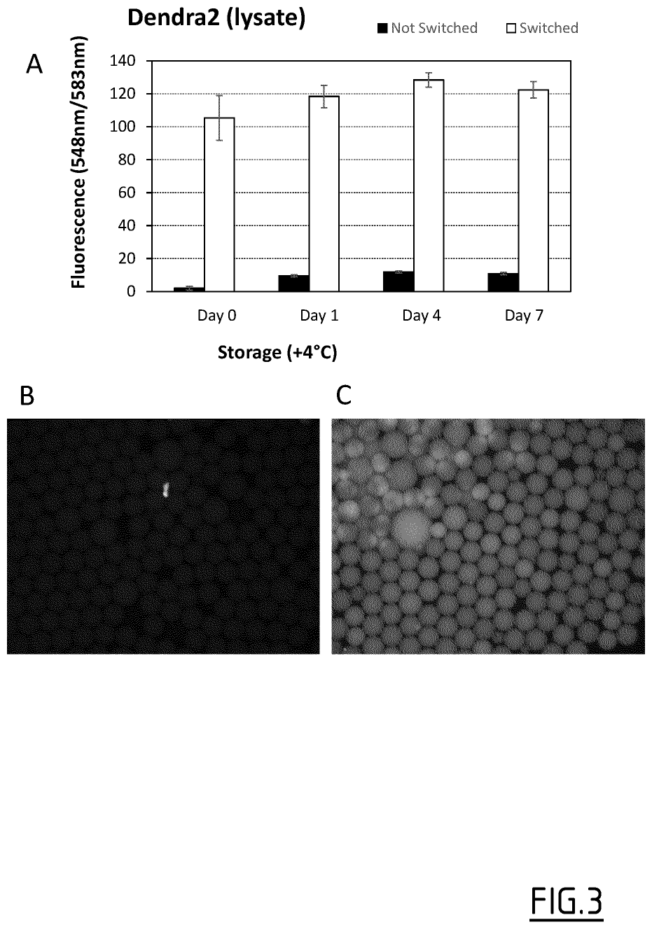

The same test was performed with the BL21-pET3-Dendra2 E. coli strain lysate instead of purified Dendra2. Indeed, there was an interest in using lysate because it is easier to prepare than pure Dendra2. Similar results were obtained: there was a significant difference of red fluorescence between before and after switch, and this difference was stable during 7 days of storage at +4° C. (FIG. 3).

Furthermore, fluorescence of droplets containing 19 μM pure Dendra2 or 3 pL of lysate was analyzed using FACS sorter. The red fluorescence intensity, in both cases, was detectable with the FACS and much higher than the fluorescence detection limit of the FACS and the on-chip sorting.

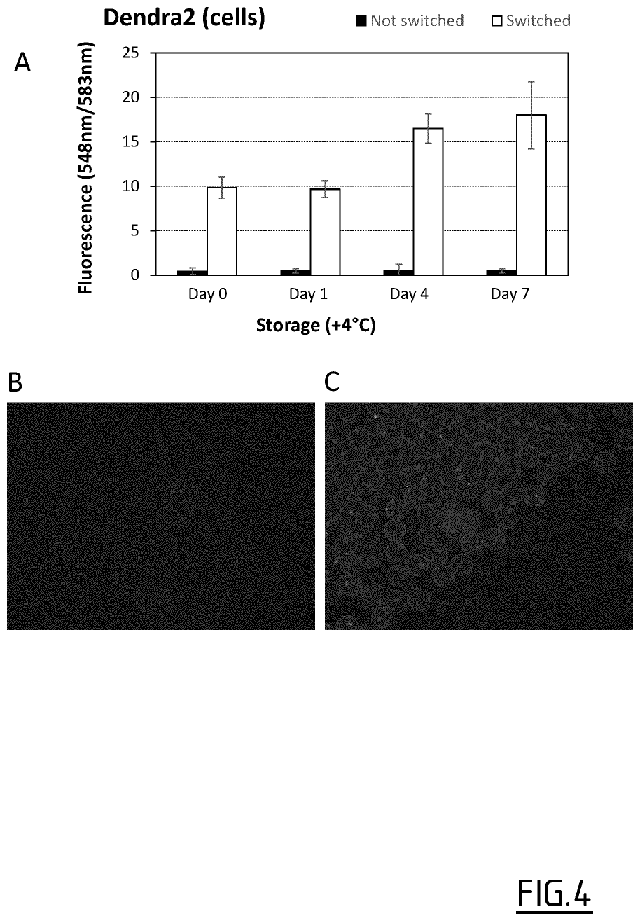

Finally, the same tests were performed directly using the BL21-pET3-Dendra E. coli strain expressing Dendra2 (FIG. 4). Indeed, in some applications, it could be of interest to use the cells as a sorting marker (ex: screening of intracellular enzymes leading to polysaccharide degradation into metabolizable monosaccharides after spontaneous cell lysis. Monosaccharides release will allow fluorescent cells to grow.

The results show that it is also possible to switch intracellular Dendra2: the red intensity was higher after switch and the difference of red fluorescence between before and after switch was maintained with the storage of Dendra2 expressing cells during at least seven days at +4° C.

To sum up, it was demonstrated that depending on the application Dendra2 PCFP could be used either in lysate, purified or directly expressed in E. coli strain. However, the fluorescence values obtained in microplate with cells at OD600 nm=5 are around 8 times lower than with lysate and pure Dendra. To prove that this point is not problematic for sorting, the minimum number of cells per droplet necessary to detect the switched droplets by FACS or by on-chip sorting was calculated. To do that, the detection threshold of the fluorescein was used, which is set to a fluorescence of 3.39 (for 5.44.10⁻³ μM) when measured in microplate. Considering that (i) BL21-pET3-Dendra E. coli cell suspension at OD600 nm=5 presents a red fluorescence of 9.8 after switch (FIG. 4A), (ii) 1 uOD corresponds to 8×10⁸ cell/mL, (iii) and a droplet of 18 μM diameter contains 3.05×10⁹ mL (3.05 pL), it was found that the detection threshold of switched BL21-pET3-Dendra E. coli strain is 4.2 cells per droplet. This threshold, calculated from fluorescence data obtained after switch in microplate at 2.5× is already very low. However, it would probably be even lower with a switch at 40×, which is more effective than the switch at 2.5×.

Leakage of PCFP from Droplets

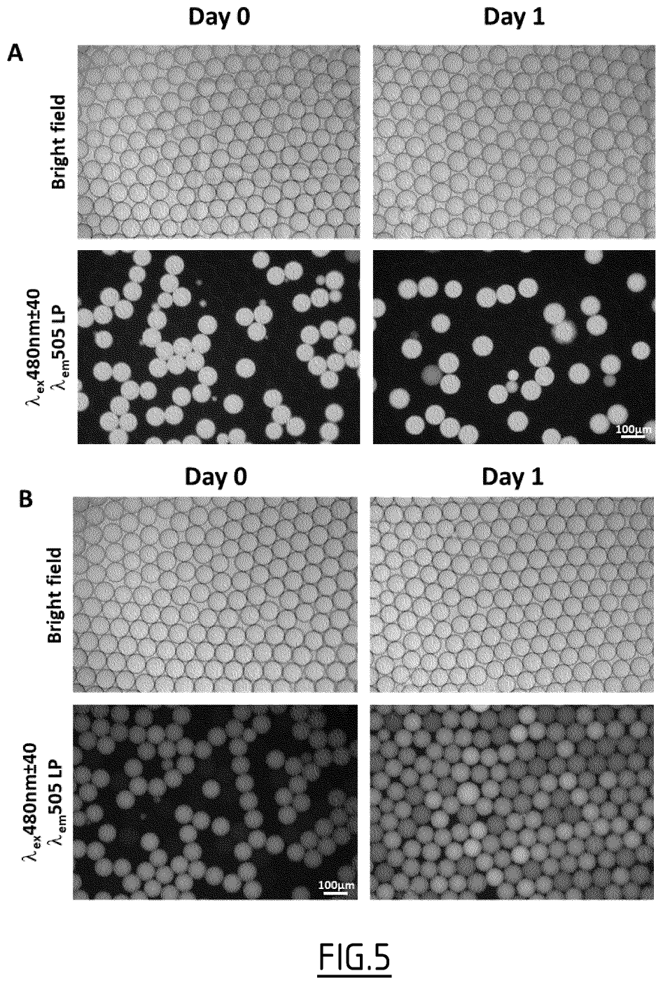

To analyse the putative leakage of purified Dendra2 protein, droplets containing water were mixed with droplets containing purified Dendra2 (FIG. 5A), and incubated the mixture for one day. As a control, the same was done with droplets containing fluorescein instead of Dendra2 (FIG. 5B). Dendra2 was the only one tested but the three PCFP have similar molecular weight.

After one day of storage at +4° C., it was observed that the Dendra2 protein remained trapped in droplets, as it was still clearly observed two population of droplets (the one with Dendra2 and the one with water, (FIG. 5A). On the contrary, when with fluorescein, all the droplets became green after one day of incubation, indicating an important leakage of fluorescein in all droplets (FIG. 5B).

In all experiments described here, water-in-oil droplets (w/o, also called single emulsion droplets) and water-in-oil-in-water droplets (w/o/w, also called double emulsion droplets) were used. Those monodisperse droplets were generated using microfluidic PDMS chips. For single emulsion droplets, the carrier phase was HFE-7500 oil supplemented with 1% RAN surfactant (RAN biotechnologies).

For double emulsion droplets production, the carrier phase was 150 mM NaCl+1% Tween 80 (Tauzin et al., 2020). Such droplets contain around 3 pL of medium.

As microorganisms were encapsulated in droplets, it was needed to control the droplet-cell occupancy. In the conditions described in the context of the invention, microorganisms were encapsulated in picoliter droplets with maximum 1 cell per droplet according to the Poisson distribution (λ 0.3), meaning that around 74% of droplets contained no cell, around 22% contained one cell and less than 4% two cells (or more, values estimated using https://www.desmos.com/calculator/j8eiciw7ds?lang=fr).

Two E. coli strains were used:
a positive control strain, which is an E. coli metagenomic clone, named F5 min_MFS, already characterized (Cecchini et al., 2013 Functional Metagenomics Reveals Novel Pathways of Prebiotic Breakdown by Human Gut Bacteria. 8 (9), 1-9; Tauzin et al., 2016 Investigating host-microbiome interactions by droplet based microfluidics) This clone has the ability to grow on minimum medium with xylo-oligo-saccharides (XOS) as carbon sources. Indeed, this fosmidic clone expresses a transporter specific for XOS and cytoplasmic CAZymes to degrade them into xylose, a carbon source usable by E. coli bacteria. This clone was transformed with an empty plasmid to confer it kanamycin resistance, in order to identify the strain in some experiments
an E. coli negative control clone, containing an empty fosmid (pCC1fos, same vector as clone F5 min_MFS). This clone was transformed with an empty plasmid to confer it ampicillin resistance, in order to identify the strain in some experiments.

The purified fluorescent Dendra2 protein was added to cell suspension just before encapsulation.

### Positive Selection of Non-Fluorescent Strains

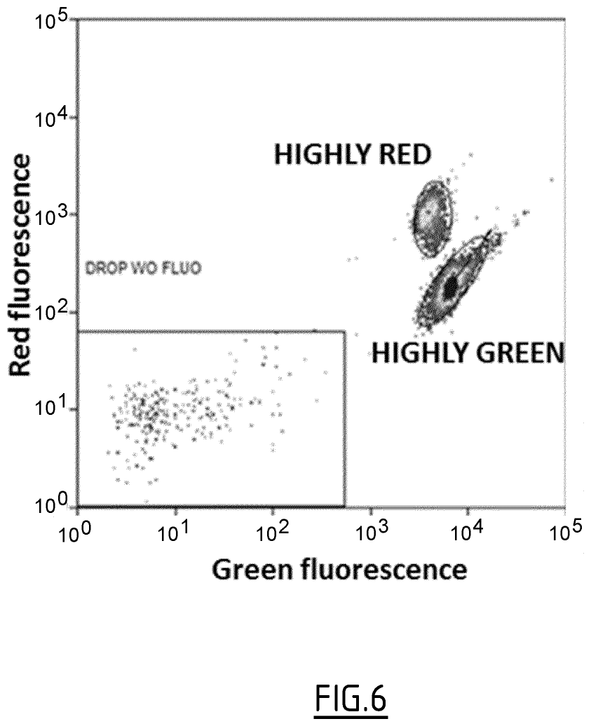

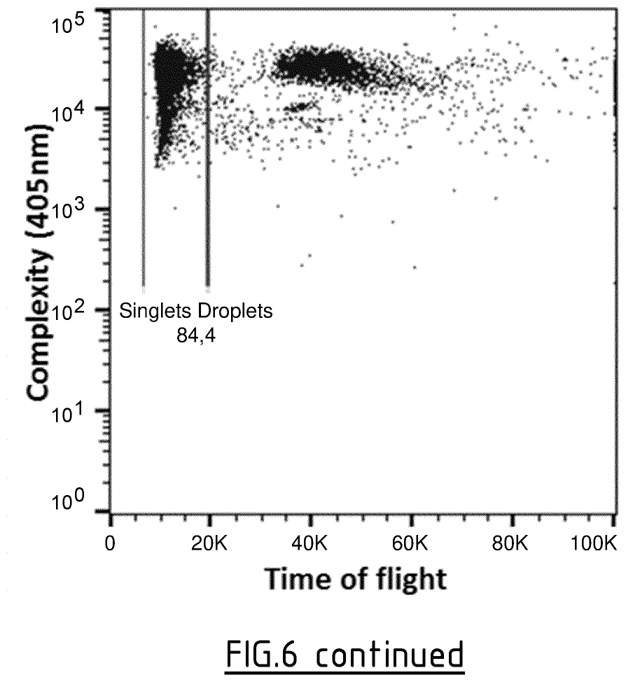

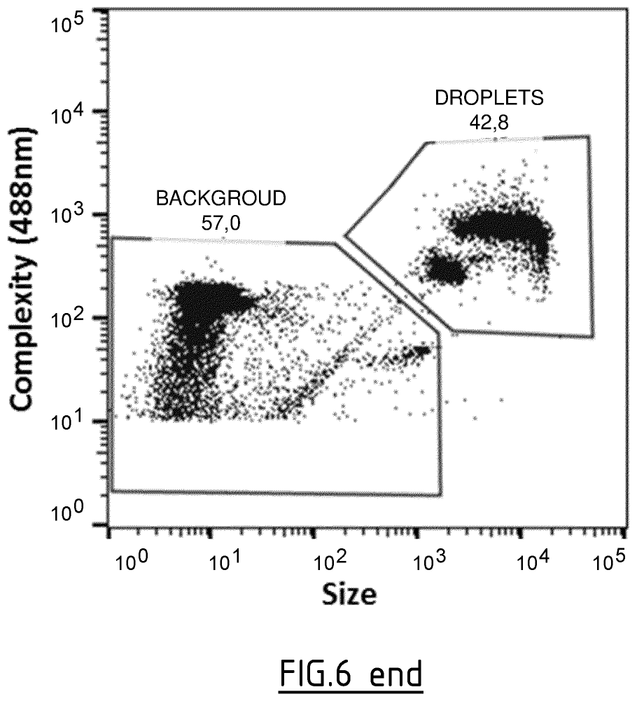

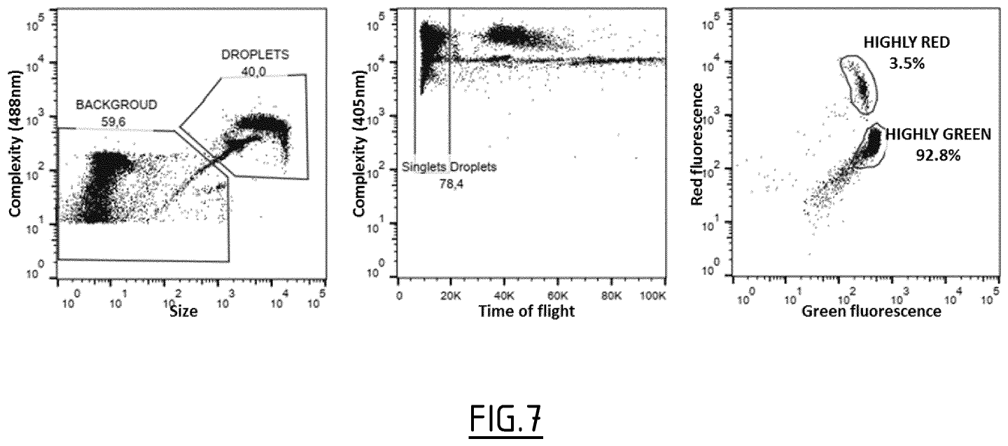

Two sets of w/o/w droplets containing purified Dendra2 and one of these two strains were prepared at high concentration (average of 85 cells per droplet). One set (containing the kanamycin resistant strain) was then switched, and both populations of w/o/w droplets mixed with different ratio and sorted (FIG. 6).

FACS identified two populations, one highly green and one highly red. These two populations were sorted and plated on selective media. As both strains exhibit different antibiotic resistances, it was possible to identify and quantify them in both droplet populations. We did the same experiment with a ratio of 95%: 5% (Table 4).

TABLE 4
Quantification of strains in both droplet populations.

| | RATIO 50%/50% | | RATIO 95%/5% | |
|---|---|---|---|---|
| Fractions | Highly green | Highly red | Highly green | Highly red |
| Clones Amp | 95.7% | 6.1% | 99.4% | 6.8% |
| resistant | | | | |
| Clones Kan | 4.3% | 93.9% | 0.6% | 93.2% |
| resistant | | | | |

For both ratio tested, in the highly green population, more than 95% of the clones were ampicillin resistant, meaning negative control strain, as expected. More importantly, in the highly red population, almost 95% of the clones were kanamycin resistant, that is to say the positive control strain, as expected too.

### Positive Selection of Non-Fluorescent Strains Grown in Droplets

The experiment was repeated with the same strains but in order to mimic a screening experiment in the real conditions of encapsulation and growth, maximum one cell per droplet was encapsulated. Before the switching step, the droplet population was incubated at 26-28° C. for bacterial growth. In this case, only the metagenomic clone used as positive control has the ability to grow on minimum medium+XOS, but not the negative control strain. One set (containing the positive control, kanamycin resistant strain) was then switched, both populations of w/o/w droplets were mixed with different ratio and sorted (FIG. 7).

As previously, the filters were applied to the mixture of w/o/w droplets to define the unique droplets.

Then droplets were sorted on their green and red fluorescence. The two populations were then plated on selective media to identify the antibiotic resistance and so, the strain (Table 5).

TABLE 5
Quantification of strains in both droplet populations after the soring
of negative and positive strains with the CultissimDrop technology

| | RATIO 98% GREEN/2% RED | |
|---|---|---|
| Fractions | Highly green | Highly red |
| Clones Amp resistant | 45.5% | / |
| Clones Kan resistant | 54.5% | 100% |

In the highly red population, all the clones were kanamycin resistant, meaning they are the positive control clone (the metagenomic clone), as expected.

### Screening for Intracellular Bacterial Enzymes, Alone and in Cocktail

Our objective was to demonstrate that the method according to the invention can be used for screening of intracellular enzymes.

To do so, a third control strain was used which was another already characterised metagenomic clone.

This one, named P4, contained a fosmid (pCC1fos, same vector as clone F5 min_MFS) with all the CAZymes needed to degrade XOS but no transporter meaning it did not have the ability to properly grow on this substrate except if some cells lysed and released CAZymes in medium to directly degrade the sugars (Cecchini et al., 2013 Functional Metagenomics Reveals Novel Pathways of Prebiotic Breakdown by Human Gut Bacteria. 8 (9), 1-9). Nevertheless, at least one cell duplication should occur in the droplet to keep a cell alive for its recovery after droplet sorting.

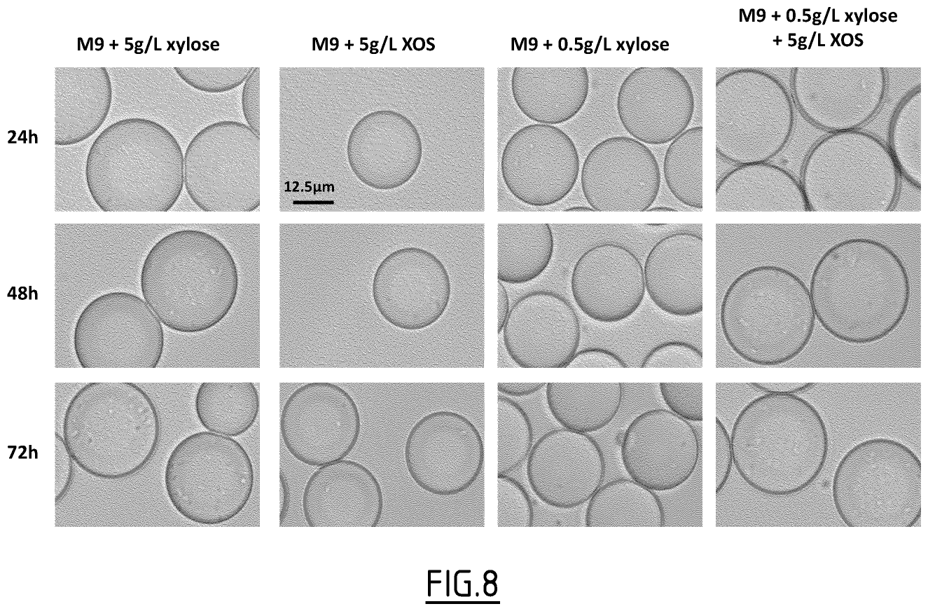

This hypothesis was tested by following bacterial growth in droplets (maximum one cell per droplet) on minimum medium containing XOS and a small amount of xylose to initiate cell growth without enabling efficient cell growth (FIG. 8).

As positive and negative controls, xylose and XOS were used respectively as carbon sources at 5 g/L.

After 72 h incubation, a proper growth was observed with 5 g/L xylose, while no growth occurred with 5 g/L XOS, as expected. The initial xylose concentration was reduced (until 0.5 g/L) to only observe a tiny growth (2 or 3 cells per droplets after 72 h incubation). At last, with 0.5 g/L xylose and 5 g/L XOS as carbon sources, a significant growth was observed, equivalent to the one on 5 g/L xylose. This indicates that spontaneous lysis occurred in the droplets, and that the method according to the invention can be used for intracellular enzyme discovery.

### Set Up for the Screening of Extracellular Fungal Enzymes and for Positive Selection of Fungal Strains

The method according to the invention was then applied to the detection of secreted enzymes. To do so, another previously characterized clone, was used. This clone was constructed in yeast Yarrowia lipolytica from a metatranscriptomic library from soil. It has the ability to grow on mannan (polymer of mannosyl residues) as it secretes a mannosidase enzyme. As negative control, a Yarrowia lipolytica strain with no mannosidase or mannanase activity was used (FIG. 9).

For the negative control strain, only a small growth was observed after 72 h incubation at 30° C., while an important growth was observed for this clone at this time. This result indicates that the method according to the invention can be used for enzyme discovery from secreting microorganisms, native or recombinant ones.

### Hit Droplet Identification and Photo-Switching Using Confocal Microscopy

After incubation of droplets to allow cell growth, the method according to the invention requires a step of identification, using optical microscope, of positive droplets (meaning droplets with cell growth or any kind of phenotype or biological process of interest visible in microscopy). Then, the fluorescence of the photoconvertible fluorescent protein, Dendra2, co-encapsulated in all droplets, has to be switched by exposing Dendra2 at a specific wavelength (Gurskaya et al., 2006). This way, the level of green fluorescence of the positive droplets decreases and red one appears (maximum emission wavelength switched from 507 nm to 573 nm with 405 nm or 488 nm wavelength exposure).

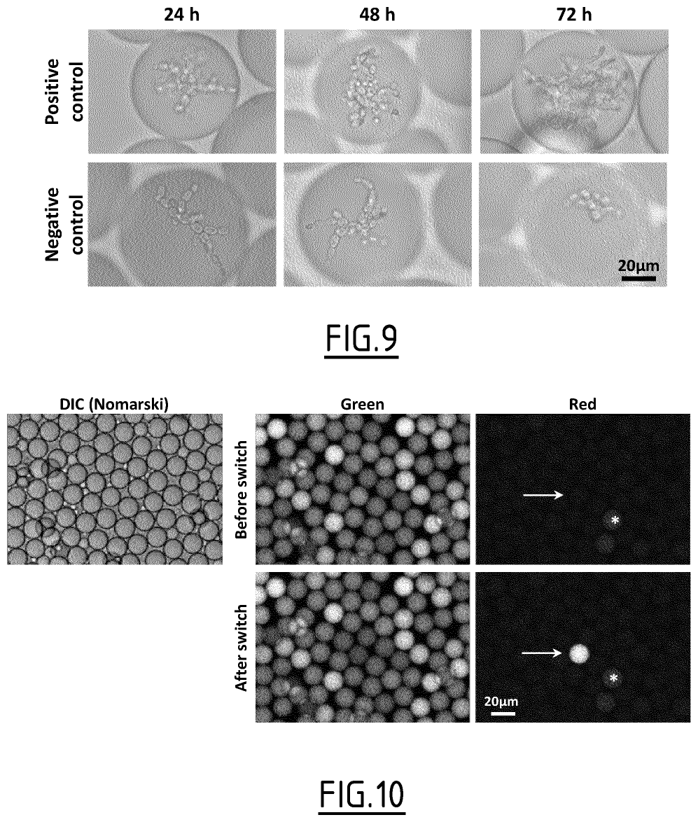

Different parameters were first tested and then set up to switch Dendra2 in one specific droplet (FIG. 10).

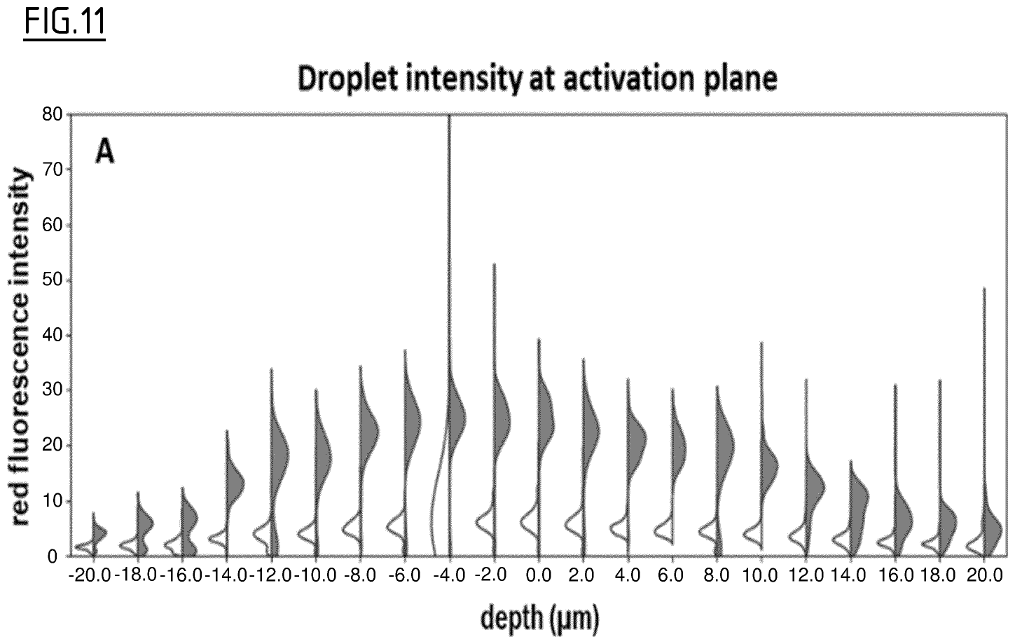

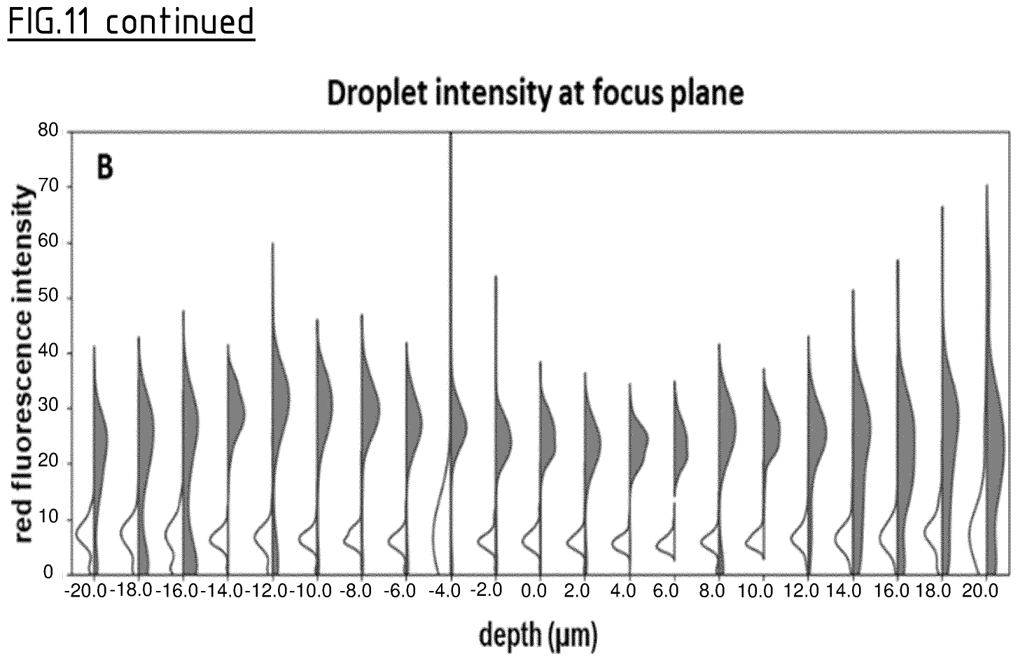

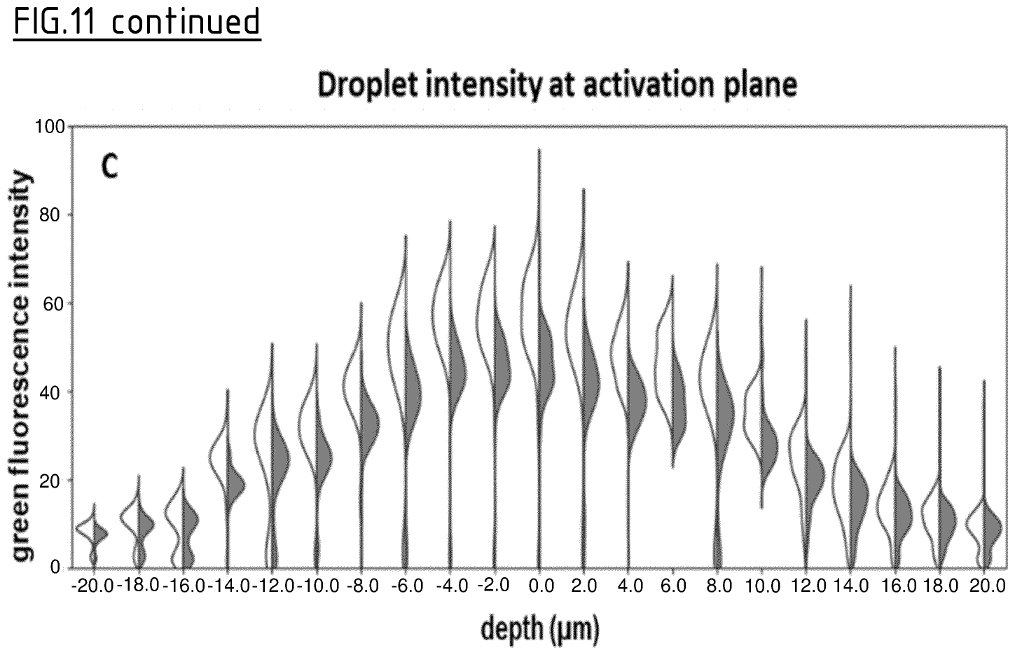

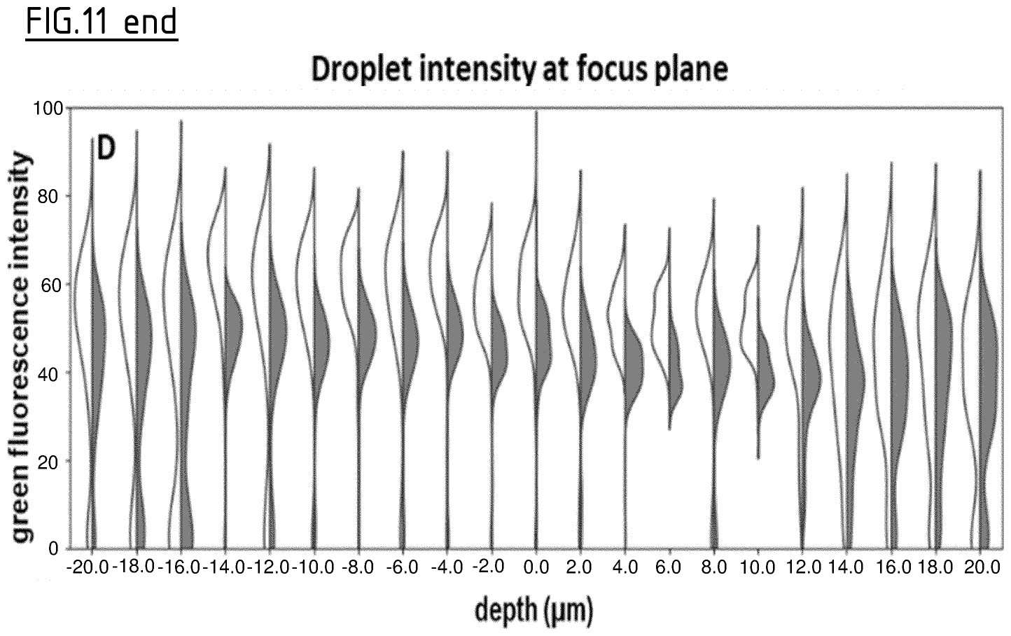

To better characterize switch efficiency, green and red fluorescence intensity of the droplet were analysed with and without switch at different planes (z dimension) of the droplet. To do so, droplets containing medium and Dendra2 protein were prepared as previously described. Before and after switch of one specific droplet, the green and the red fluorescence intensity was measured at the activation plane and at focus plane. Measurements were done on different drops for every planes (FIG. 11).

The focus plane was defined as the plane “zero”. As expected, it was observed that the green fluorescence intensity of the switched droplets was slightly lower than for the unswitched droplets. This was true whatever the activation plane was and when measured at this activation plane and at the focus plane.

Besides, the more the activation plane was far from the focus one, the more the green and red fluorescence intensities measured at the activation planes decreased. Nevertheless, for all the activation planes from −12 μm to +12 μm, the switching process allowed to significantly increase the red fluorescence intensity of the switched population and distinguished it from the unswitched one at both planes.

These results demonstrate that if a loss of focus would occur during the switching step:
it wouldn't affect the efficiency of the switch and then the activation step (i.e. labelling step),
the green and red populations would exhibit clearly different red fluorescence intensities, meaning it wouldn't affect the sorting step.

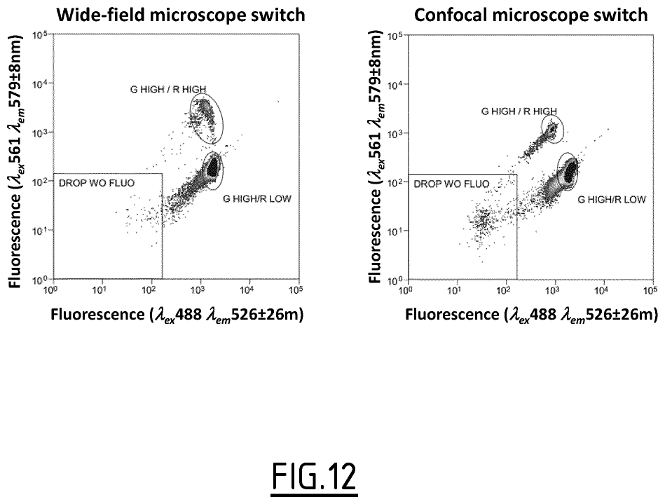

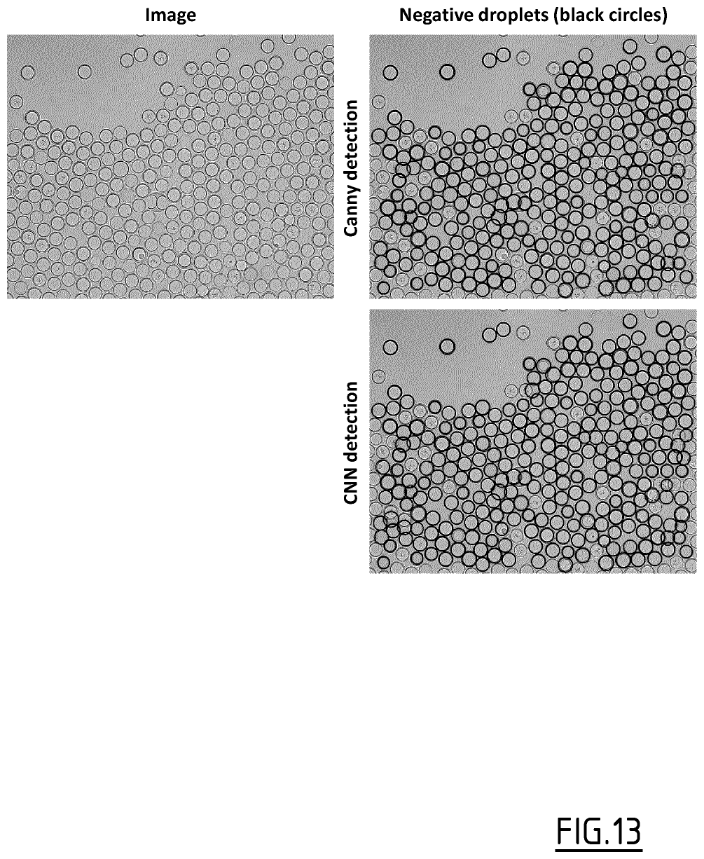

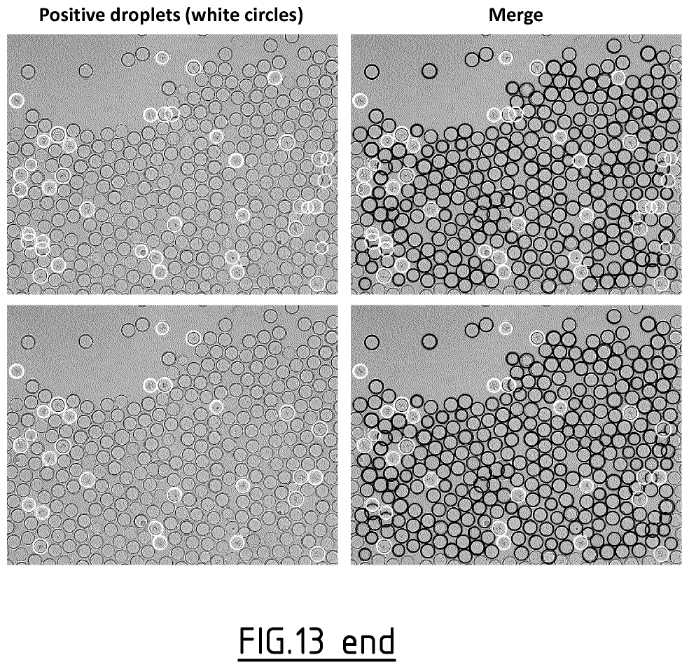

Previous sorting has exclusively been done with droplets switched using wide-field microscopy. To verify that droplets switched using confocal microscopy could be sorted with FACS with the same filters, two sets of droplets were prepared, one containing the positive control, E. coli metagenomic clone, named F5 min_MFS, and the other containing the negative control on selective medium with XOS as carbon sources. After 6-day incubation, the Dendra2 protein in droplets were switched with the positive control clone, either with wide-field microscope or with laser scanning confocal microscope, and then the obtained droplets were analysed with FACS (FIG. 12). For the test with droplets switched with wide-field microscopy, a ratio of switched/unswitched of 1.3/98.7 was used whereas for the test with droplets switched with confocal microscopy, a ratio of 6.2/93.8 was used. After analysis of green/red fluorescence intensity of the mixture of droplets with FACS, it was found, in both cases, the two populations with the good ratio. Furthermore, as expected, the green population of droplets exhibited the same levels of fluorescence. The red population seemed to be much more compact and the separation of the two populations was more efficient, after confocal microscopy-based switch.

As laser was applied on droplet, meaning on cells we wanted to collect and grow, the potential effect of iterations of laser was checked on cell viability. To do so, from no to 80 iterations of the laser were applied on droplet suspensions. After switch, cells were collected by filtration and quantified on agar plate.

It was found out that cell viability did not change whatever the number of laser iterations was.

### Droplet Classification Improvement

The inventors also studied the reduction of false positive by using a Convolutional Neural Network compared to an algorithm using an edge detector (canny filter) to detect entities of interest.

The CNN architecture takes into input a 64 by 64 pixels image and output a number between 0 and 1, with 1 being a positive droplet and 0 a negative one.

As known per se, the convolutional neural network alternates between a convolution layer and a down-sampling layer.

The CNN architecture here comprises two convolutional/down-sampling pairs, followed by a fully connected layer that classifies output with one label per node. The network used for the test comprises here six layers, in order: a first 2D convolution layer, a first max pooling operation for 2D data, a second 2D convolution layer, a second max pooling operation for 2D data, a flatten operation and a dense operation.

The classification with an edge filter, called here method “canny”, is done in two steps. First the canny edge detector filter is applied on the droplet images. Secondly, on this edge image, the mean intensity of a circular region smaller than the droplet size and centered on the droplet is measured. If the mean intensity is higher than a threshold value, the droplet is considered as positive.

Bacterial growth was studied on a dataset of 17,491 droplets (1,372 positives and 16,119 negatives) including out-of-focus and superposed droplets (Table 6). With the method “canny”, a canny filter then a mean intensity threshold (here 0.2 or 0.1) was applied to the droplets. With the second method “CNN network”, the convolutional neural network had been trained to detect bacterial growth.

TABLE 6
Detection of bacterial growth.

| Method | False positive | False negative |
|---|---|---|
| Canny (0.2) | 7.80% | 19.40% |
| Canny (0.1) | 24.00% | 3.80% |
| CNN network | 0.16% | 1.70% |

Table 6 shows that using a convolutional neural network according to the invention greatly reduces the percentage of false positives and false negatives, compared to using an algorithm with an edge detector.

1. A method of screening of at least one phenotype or biological process in a high throughput droplet micro-to-milli-fluidic system, said method comprising the following steps of:
(a) generating a droplet batch in a carrier fluid to form a plurality of individual bioreactors, each droplet containing a photoconvertible or a photoactivable fluorescent protein and broth media, solution or buffer, at least one of the droplets containing one or several entity (ies),
(b) incubating said droplets over time,
(c) detecting among said droplets at least one droplet of interest by imaging them,
(d) labelling said detected droplets by switching the fluorescence of the photoconvertible or a photoactivable fluorescent protein,
wherein said detecting and labelling steps are conducted by an automated electronic processing system, and
(e) selectively recovering the droplets using the label of the droplet.

2. The method according to claim 1, wherein said method further comprises:
(f) recovering the entities; and optionally
(g) submitting said entities to biological analysis.

3. The method according to claim 2, wherein the biological analysis of step (g) comprises but is not limited to the DNA sequencing, taxonomical and functional annotation of the genes, phenotypic characterization or enzymatic activity characterization of the recovered entities.

4. The method according to claim 1, wherein the volume of each droplet is from pL to μL.

5. The method according to claim 1, wherein the entities are selected from the group consisting of prokaryotic cells, eukaryotic cells, phages, viruses, plasmids, proteins (including enzymes) and self-replicating RNA.

6. The method according to claim 1, wherein the entities are selected from the group consisting of bacteria, archea, unicellular eukaryotes, cell lines derived from multicellular eukaryotes, microorganisms communities, small multicellular organisms, terrestrial fresh water and marine samples, extraterrestrial samples, clinical samples, proteins and enzymes.

7. The method according to claim 1, wherein said photoconvertible or photoactivable fluorescent protein is chosen from the fluorescent proteins Dendra2, PA-GFP or PATagRFP and is encapsulated in a purified form, or under the form of a bacterial lysate, or under the form of a microbial cell producing the photoconvertible or photoactivable fluorescent protein.

8. The method according to claim 1, wherein the droplets are chosen from water/oil/water (w/o/w) double-emulsion droplets and water/oil (w/o) single-emulsion droplets.

9. The method according to claim 1, wherein the detecting step comprises the following sub-steps, implemented by the electronic processing system, of:
(c1) obtaining an image portion of at least some of the droplets of the droplet batch, said image portion representing a single droplet; and
(c2) classifying said droplet by using an artificial intelligence algorithm, the algorithm having as input variable the image portion and as output variable a class of the droplet, the class of the droplet being representative of the phenotype or biological process resulting from the activity of the at least one entity encapsulated within.

10. The method according to claim 9, wherein the step of obtaining the image portion comprises the steps of:
(c1a) acquiring at least one image of a plurality of droplets of the droplet batch under the form of droplet monolayers in an observation chamber, the at least one image being acquired using a camera; and
(c1b) segmenting the at least one image into at least one image portion by applying an edge detector filter and performing a circular Hough transform.

11. The method according to claim 9, wherein the artificial intelligence algorithm is an artificial neural network.

12. The method according to claim 9, wherein the detecting step further comprises the following sub-step of:
(c3) determining the coordinates of each droplet belonging to a class corresponding to the desired phenotype or biological process of interest.

13. The method according to claim 12, wherein the labelling step comprises the following sub-steps of:
(d1) acquiring the or each image;
(d2) defining a region of interest for each droplet of interest, the region of interest being centered on the coordinates of the related droplet of interest; and
(d3) switching the fluorescence of the photoconvertible or photoactivable fluorescent protein in the droplets on all regions of interest.

14. The method according to claim 1, wherein the labeling step is performed by laser-scanning microscopy.

15. The method according to claim 14, further comprising a step of correcting droplet coordinates by correlating a camera image and a confocal image of a same zone, measuring a shift between said camera image and said confocal image; and correcting the coordinates during the image acquirement sub-step, based on the measured shift.

16. The method according to claim 1, wherein the at least one phenotype or biological process is self-replicating ability of microbial cells.

17. The method according to claim 6, wherein the unicellular eukaryotes are selected from the group consisting of yeast, algae, and slime molds.

18. The method according to claim 6, wherein the eukaryotes are plants or animals.

19. The method according to claim 11, wherein the artificial neural network is a convolutional neural network.

20. The method according to claim 14 wherein the laser-scanning microscopy is laser-scanning confocal microscopy.
